(19) United States
(12) Patent Application Publication
(10) Pub. No.: US 2024/0382643 A1
(43) Pub. Date: Nov. 21, 2024

(54) AROMA DEVICE APPLICATIONS

(71) Applicant: General Mills Inc., Minneapolis, MN (US)

(72) Inventors: Scott Daniel Klimek, Minneapolis, MN (US); Janavi Kumar, Plymouth, MN (US); Christine Ng, MInneapolis, MN (US)

(21) Appl. No.: 18/661,892

(22) Filed: May 13, 2024

Related U.S. Application Data
(60) Provisional application No. 63/466,570, filed on May 15, 2023.

(51) Int. Cl. A61L 9/12; A61L 9/01

(52) U.S. Cl. CPC A61L 9/12; A61L 9/01; A61L 2209/11

(57) ABSTRACT

Methods of using an aroma device associated with food experiences are disclosed. A method can include obtaining food-product data corresponding to a food product; based on such data, selecting an aroma profile for the food product; emitting an aroma based on the profile; obtaining modification data; and generating a new aroma profile for the food product based on the modification data. A method can include decoupling two or more aromas based on human and pet aroma sensitivity and/or preferences. Systems configured to use an aroma device are also provided.

### FIELD

The present disclosure relates to uses of an aroma device, and more specifically to uses of an aroma device associated with food experiences.

### BACKGROUND

An aroma device can include an apparatus that can emit a chemical composition (e.g., composition of alcohols, aldehydes, acids, esters, terpenes, pyrazines, and furans) proximate a user. Food product development can include providing a range of food samples to a large number of testers (e.g., humans or animals) who handle and consume the food samples and acquiring data from the testers that may be useful for improving existing food products and/or developing new food products.

### SUMMARY

According to embodiments of the present disclosure, a method for use with an aroma-emitting device can include obtaining food-product data corresponding to a food product; selecting, based on the food-product data, an aroma profile for the food product; emitting, by the aroma-emitting device, an aroma based on the aroma profile; obtaining modification data corresponding to an instruction to modify the aroma; and generating a new aroma profile for the food product based on the modification data.

According to embodiments of the present disclosure, the system can include an aroma-emitting device; a controller including one or more processors; and one or more computer-readable storage media storing program instructions which, when executed by the one or more processors, can cause the one or more processors to perform a method. The method can include obtaining food-product data corresponding to a food product; selecting, based on the food-product data, an aroma profile for the food product; obtaining an indication that an aroma based on the aroma profile is emitted by the aroma-emitting device; obtaining modification data corresponding to an instruction to modify the aroma; and generating a new aroma profile for the food product based on the modification data.

In some embodiments, food-product data can include information such as identifying information for a food product, information regarding ingredients of a food-product, etc. In some embodiments, selecting an aroma profile for a food product can be based, at least in part, on food-product data. In some embodiments, selecting an aroma profile for a food product can be based, at least in part, on user data (e.g., a stored user profile including user identification information, previous user preferences/selections, biometric data of the user, information about a user's physical condition and/or activity (e.g., heart rate, respiration, body temperature, physical movements, and the like).

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 depicts example hardware components of example aroma devices, according to embodiments of the present disclosure.

FIG. 2 depicts an example aroma device configuration, according to embodiments of the present disclosure.

FIG. 3 depicts an example aroma device configuration, according to embodiments of the present disclosure.

FIG. 4 depicts an example use of an aroma device, according to embodiments of the present disclosure.

FIG. 5 depicts an example aroma device configuration, according to embodiments of the present disclosure.

FIG. 6 depicts example aroma device applications, according to embodiments of the present disclosure.

FIG. 7 depicts an example method for using an aroma device, according to embodiments of the present disclosure.

FIG. 8 depicts an example use of an aroma device, according to embodiments of the present disclosure.

FIG. 9 depicts an example use of an aroma device, according to embodiments of the present disclosure.

FIG. 10 depicts an example use of an aroma device, according to embodiments of the present disclosure.

FIG. 11 depicts an example computing device that can be used in accordance with embodiments of the present disclosure.

While the disclosed subject matter is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications (e.g., rearranged and/or reversed method operations), equivalents, and alternatives falling within the scope of the disclosure as defined by the appended claims.

### DETAILED DESCRIPTION

The present disclosure relates to uses of an aroma device to affect food experiences.

An aroma/aroma profile can include: a combination of distinct aromatic (volatiles); a chemical composition (e.g., composition of alcohols, aldehydes, acids, esters, terpenes, pyrazines, and furans); components that influence a sensory perception of a flavor or fragrance; and/or a characteristic or fingerprint of an odor. An aroma/aroma profile can represent a set of characteristics for a particular food.

An aroma camp can include a descriptive category of aromas/aroma profiles. Examples include lower-resolution categories, such as a fruit category or a cheese category of aromas/aroma profiles, and higher-resolution categories, such as an apple category or a gala apple category of aroma profiles.

Embodiments of the present disclosure can include applications such as:

Technology, hardware, and methods can be used to rapidly evaluate aroma designs that can enhance enjoyment of food without making food prototypes. Electric-based (or chemical-based, or electric-chemical based) aroma creation technologies can be used to create simulated aromas to test effects in consumers. Novel aroma combinations may be identified to elevate enjoyment of food by enhancing retro-nasal taste without the use of macros and micronutrients consumers may want to avoid (e.g. fat, sugar, salt), or by being able to trial a larger set of combinations due to enhanced efficiency in testing flavor combinations according to embodiments of the present disclosure.

Digitally activated aromatic solutions can be used to enhance the speed and learning process of screening for optimal product design. One example of a hardware/software technology that can deliver digitally activated aromas (scent) is the OVR Technology (ovrtechnology.com). It uses a piezoelectric-based odor reproduction system. Technology such as this can be leveraged to design a system to help food companies to rapidly identify optimal, novel, or unique combination of aromas to create elevated food experiences without having to create many food prototypes for consumers to taste.

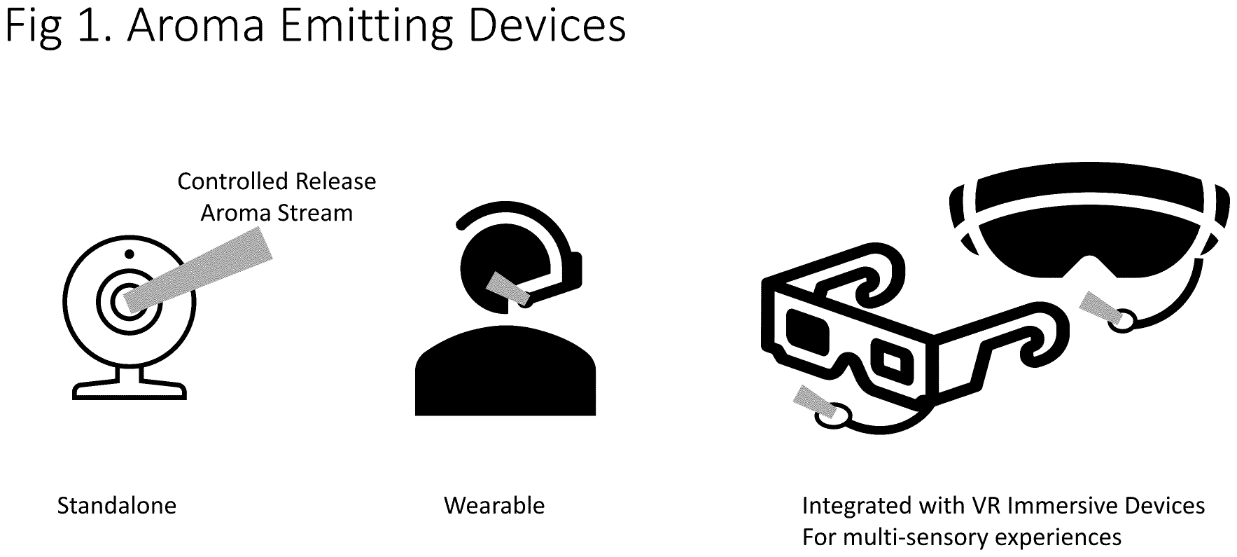

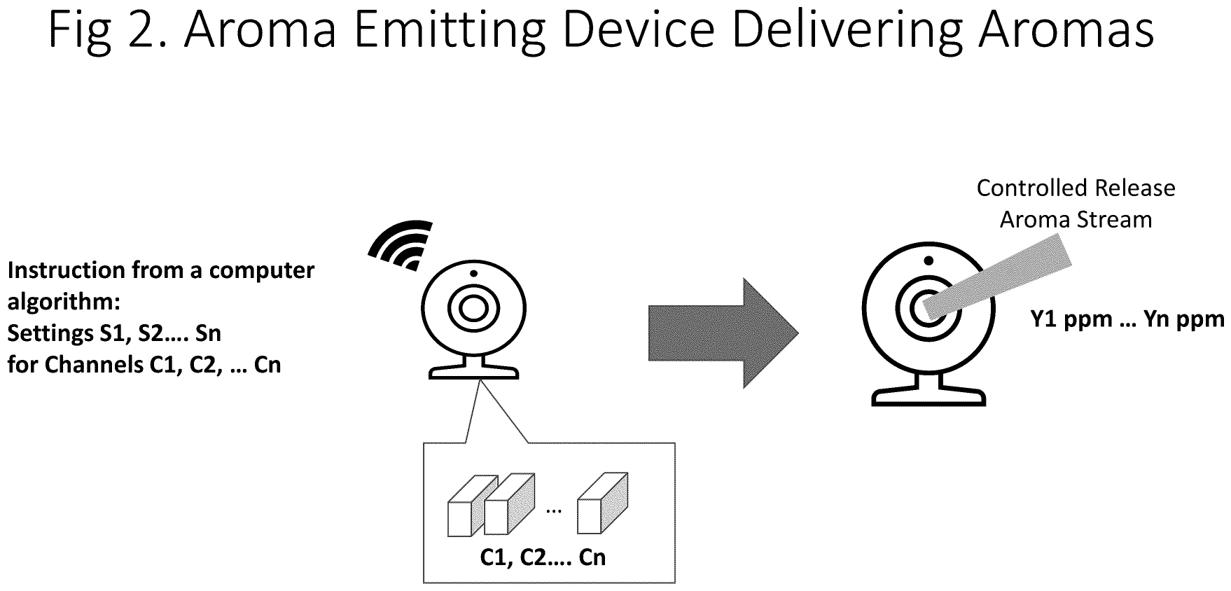

As shown in FIG. 1, embodiments of the present disclosure include an aroma device integrated with an immersive device. An immersive device can include a device such as a mobile or wearable device (e.g., headset/goggles, phone, tablet, notebook computer, and the like) configured to provide a user with an Augmented Reality (AR), Virtual Reality (VR), and/or Mixed Reality (MR) experience. An AR experience can include an overlay by an immersive device of digital content onto a view of a real-world environment. A VR experience can include an immersive device providing a virtual environment in which a user can experience situations that mimic real life or creative imaginary environments. A MR experience can combine AR and VR by capturing aspects (e.g., images, sounds, etc.) of a real-world environment, combining such aspects with virtual objects, and displaying/presenting both to a user.

### Data Obtained by the Aroma Device

In some embodiments, example devices that can provide user data to the aroma device can include an electroencephalogram (EEG), galvanic skin response (GSR), heart rate monitor, and the like. The aroma device can be configured to obtain data from a user in a variety of ways (e.g., survey, wearable device, image/video data); thus, the aroma device can generate aroma profiles based on input from a user that is voluntary/conscious (e.g., a user states, “I like this.”) and/or involuntary/subconscious (e.g., a user's heart rate or body temperature changes). Additionally, the aroma device can store and/or analyze correlations between such input and selected and/or emitted aroma/aroma profiles.

### Using an Aroma Device

A user device (e.g., smart phone, tablet, smart watch) can include a graphical user interface associated with software that permits a user to manipulate aspects of an aroma stream emitted from an aroma device. In an example, the graphical user interface can include a graphic such as a dial that permits a user to increase and/or decrease an aspect such as an intensity of the aroma. The configuration can permit a user to eat a food product having a lower degree of sweetness and simultaneously perceive that the food product has a higher degree of sweetness. The user's perception can be due to the user selecting to increase a sweetness level of an aroma stream emitted from the aroma device.

In some instances, an aroma device can automatically select an aroma profile for a user based on user input data. For example, at a first time, a user can identify a preferred aroma profile associated with eating a food, such as popcorn. The user can store data corresponding to the preferred aroma profile and the food in a user profile. At a second time, subsequent to the first time, the user can provide the stored data to the aroma device (e.g., the user can present a QR code corresponding to the stored data to a scanner of the aroma device). In response, the aroma device can select the preferred aroma profile to be emitted while the user consumes popcorn. Continuing with this example, at a later time, a second user can provide additional data corresponding to the second user's preferred aroma profile for popcorn or a different food. In response, the aroma device can select the second user's preferred aroma profile to be emitted while the second user consumes the popcorn or a different food.

Accordingly, in some embodiments, a user can manually modify (e.g., by a user control, such as a dial on a graphical user interface) an aroma profile emitted from the aroma device. In some embodiments, the aroma device can automatically (e.g., autonomously) modify an aroma profile emitted from the aroma device. In some embodiments, a user can manually select an aroma profile to be emitted from the aroma device. In some embodiments, the aroma device can automatically select an aroma profile to be emitted from the aroma device. In some embodiments, the ability of the aroma device to autonomously collect data from a user and select/emit an aroma/aroma profile can improve results by requiring less direct attention/handling by a user.

### Applications

Aroma is shown to have impacts on emotion/mood/memory. Embodiments of the present disclosure can allow users to instantly adjust the food perception of a food product, such as customizing, augmenting or tailoring to mood by changing aroma inhaled (in the moment) while eating the food through interaction with the aroma emitting device (consciously, or automated with an algorithm), without physically creating a new food product. Augmenting the sensorial experience possible with a single food item.

### Tailoring to Mood

In an example, an aroma device can obtain information about a user's emotion/mood and use such information as at least one basis for selecting and/or emitting an aroma/aroma profile. The aroma device can obtain such information in a variety of ways, such as by a user survey on a graphical user interface associated with the aroma device; by a wearable device (e.g., a smartwatch/activity tracking device); etc. In an example, based on data indicating that a user is stressed/uncomfortable, the aroma device can select an aroma profile deemed to have a calming/comforting effect (e.g., an aroma profile including lavender). Additionally, the aroma device can store and/or analyze correlations between such emotion/mood data and the selected and/or emitted aroma/aroma profile.

### Health/wellness Applications

In some embodiments, an aroma device can obtain user health/wellness information (e.g., blood sugar level, weight, etc.), and use such information as at least one basis for selecting and/or emitting an aroma/aroma profile. In an example, for a user attempting to manage the user's glucose levels, an aroma device can select and/or emit an aroma/aroma profile that can encourage the user to consume more food or less food, as needed, for glucose-level management. In this example, the selection and/or emission by the aroma device can be based on blood sugar data obtained from the user. Accordingly, embodiments of the present disclosure can provide a non-caloric approach to adjusting food preference and/or consumption patterns to influence a certain desired outcome (e.g., without adding sugar or removing sugar, embodiments of the present disclosure can encourage a user to eat more or eat less). Additionally, the aroma device can store and/or analyze correlations between health/wellness data and a selected and/or emitted aroma/aroma profile.

### Screening for Research and Development

Start with a base food like a vanilla yogurt and, by the aroma device, expose the user to a variety of aroma profiles while the user consumes the base food, and obtain feedback from the user (e.g., a degree of liking of a combination, a level of expectation regarding the combination, etc.).

An example food test can include a user wearing one or more monitoring devices (e.g., a heart rate monitor). The user can be provided a food sample to consume while an aroma device emits an aroma that the user experiences while consuming the food. The aroma device can obtain data from the user via the one or more monitoring devices and/or survey data (e.g., a user's degree of liking the food sample and aroma combination; whether the user would purchase a food product corresponding to the food sample and aroma combination; whether the food sample and aroma combination is congruent, or is a good match; a user's perceived intensity; a user's perceived characteristics/taste regarding the food sample and aroma combination (e.g., whether it tastes artificial, tastes sweet, etc.); and the like). In some instances, the user device can obtain data based on the user's selection (e.g., a user manually adjusting an emitted aroma to a preferred level of sweetness). In some instances, the aroma device can prompt a user to provide feedback (e.g., an aroma device can modify an aroma and prompt a user to respond whether the modification improves or worsens the food sample and aroma combination). The aroma device can make subsequent modifications to the emitted aroma based on such user responses.

In another example, a user can be provided a baked sample of a food, as opposed to a fried sample of the food. The baked sample can have less fatty aroma than the fried sample. The user can adjust an aroma profile emitted by an aroma device to identify a preferred flavor combination with the baked food (e.g., a ranch flavor or an imitation of volatiles commonly associated with fried food such as a blend of aldehydes, ketones, and fatty acids). Based on aroma profiles selected by users, an entity can identify flavor combinations that can be developed into corresponding food products.

In some embodiments, an aroma device can be configured to provide a set of aromas for a user's selection, obtain feedback data based on the set of aromas, and based on the feedback data, perform operations such as analyzing the feedback data for generating a new aroma profile and/or providing a new aroma for the user's selection, transmitting the feedback data (e.g., to a separate computing device) to be analyzed for generating a new aroma profile and/or providing a new aroma for the user's selection, and/or generating a new aroma profile and/or providing a new aroma for the user's selection.

### Additional Benefits

Embodiments of the present disclosure can provide additional benefits such as:

A user can experience a food without consuming the food (e.g., a user allergic to peanut butter can experience eating peanut butter without actually doing so).

Generated aroma profiles can be used for market research and product design.

Embodiments of the present disclosure can permit a relatively large number of iterations of food testing/sampling without the need to generate/provide large varieties of food samples. For example, a vanilla cupcake can be tested with 10 types of strawberry flavors by providing one type of cupcake and 10 aroma profiles to be tested with the cupcake, as opposed to providing 10 types of cupcakes, each having a different strawberry flavor. Embodiments of the present disclosure can facilitate an increased number of food samples tested by a user. For example, testing of a high-calorie food can be mimicked by a combination of a low-calorie food and an aroma emitted by the aroma device. Thus, a user may be able to consume and provide feedback on a larger quantity of the low-calorie food samples than the user would be able to consume and provide feedback on with high-calorie food samples.

Embodiments can provide more data with increased accuracy regarding food testing. In an example, population-level insights (over 100s/1000s of consumers), can be efficiently obtained, according to embodiments of the present disclosure. Embodiments of the present disclosure can allow for rapid and more accurate feedback on product design options (help consumers articulate insights about aroma) and improve congruency (consumer expectations vs delivery of flavor).

Testing data can be used to develop food products corresponding to various food sample/aroma combinations.

Several applications covering benefits of rapid product design, effective and more efficient consumer testing, delivering more joy to consumers (including pet parents and pets) by delivering augmented experiences, enabling solutions for improved consumer outcomes related to weight-loss, health and wellness, etc.
1. Broad consumer testing
2. Co-Creation with consumers
3. Augmented Brand Experiences
4. Weight-Loss
5. Wellness & Health
6. Individualized Experiences
7. Flavor Masking/Shelf-life Extension/Food Waste/Cost Saving
8. 3D printed food
9. Decoupled Pet food aroma adjustment for pet or pet parents

Embodiments of the present disclosure can permit a user to eat a food product having a lower degree of sweetness and simultaneously perceive that the food product has a higher degree of sweetness. The user's perception can be due to the user selecting to increase a sweetness level of an aroma stream emitted from the aroma device.

Based on data associated with user adjustments of an aroma profile, an entity, such as a food company, can identify an aroma profile most preferred by users (e.g., an aroma profile that provides the level of sweetness preferred by most users). By identifying such an aroma profile, the entity can develop a corresponding food product having ingredients that can provide a corresponding (e.g., identical or substantially similar) aroma when consumed.

In some embodiments, data related to aroma profiles can be used to design aroma experiences in public places, such as a physical or virtual retail store, to enhance a shopping experience and/or to provide a preview of a product experience. For example, because human aroma sensitivity is generally lower than their pets' aroma sensitivity, an aroma device in a retail store can be used to express an aroma that is adjusted for pet enjoyment at levels that are less intense (or absent) relative to an aroma that is adjusted for acceptance and/or enjoyment by a pet parent. Such an aroma experience can preview a pet parent's experience with a pet food designed to have an aroma that is acceptable and/or pleasing to a pet parent, while still providing aromas that their pet will enjoy at levels that do not create significant pet parent discomfort.

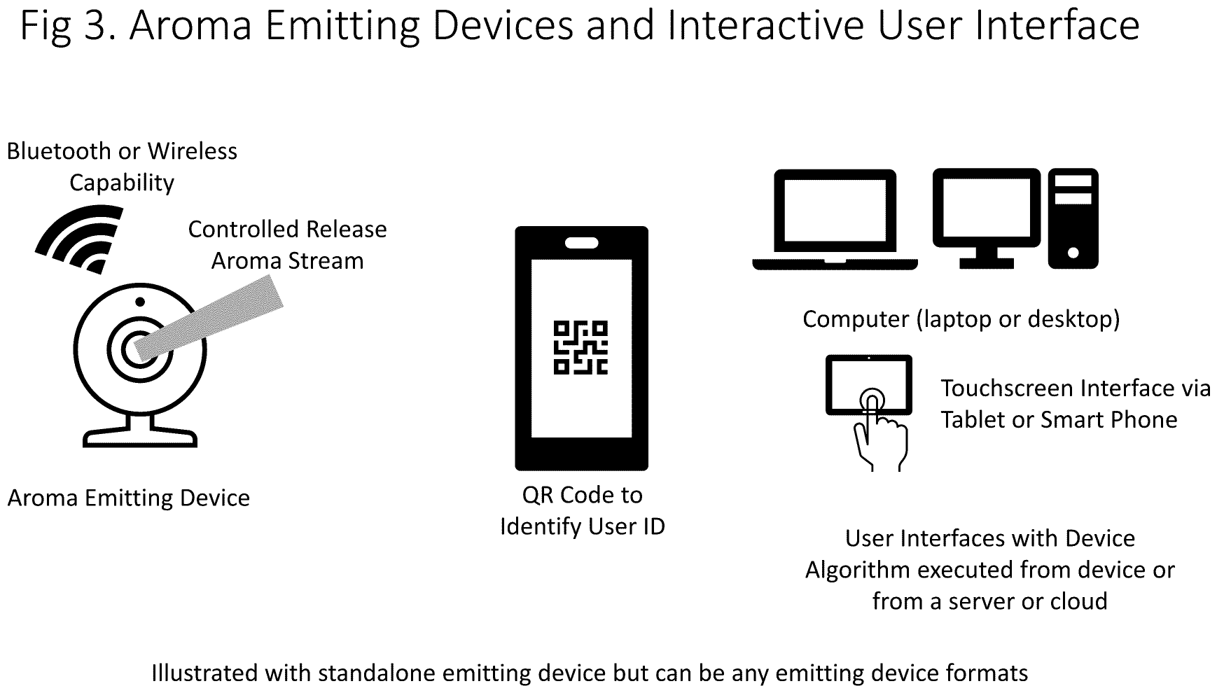

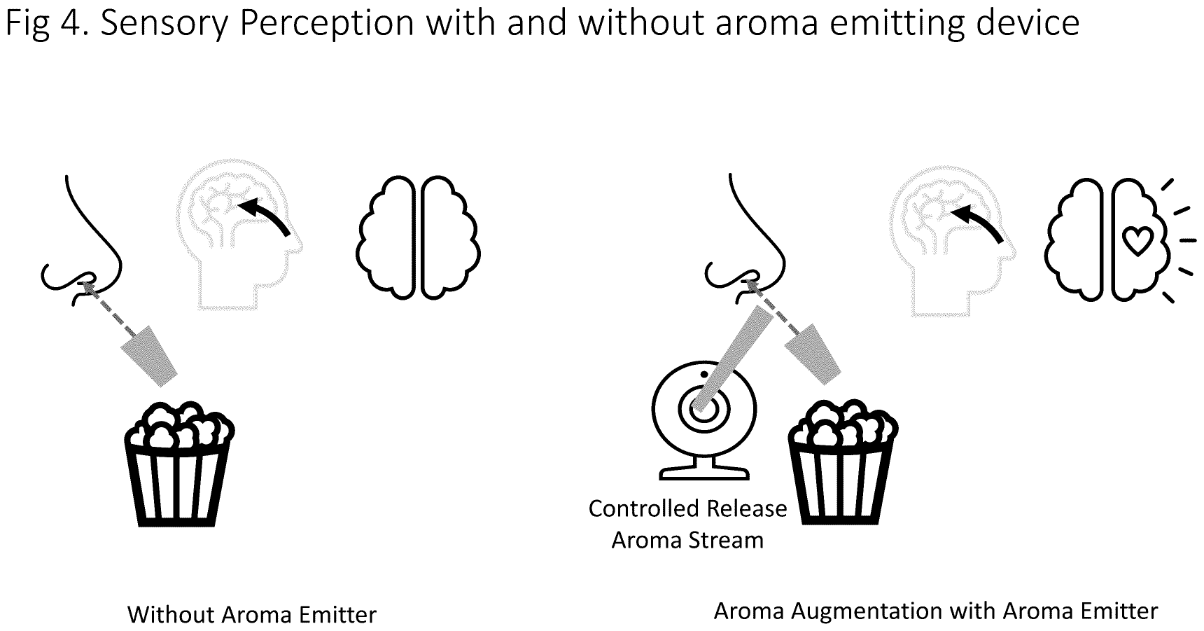

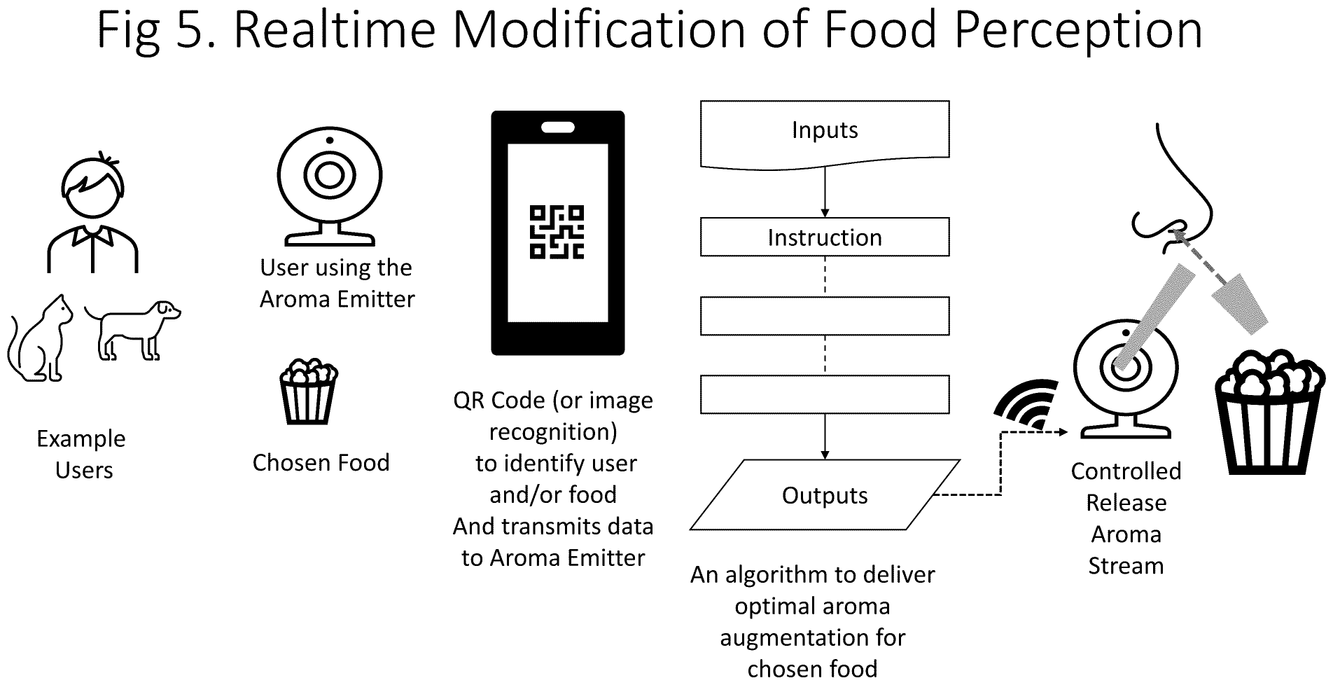

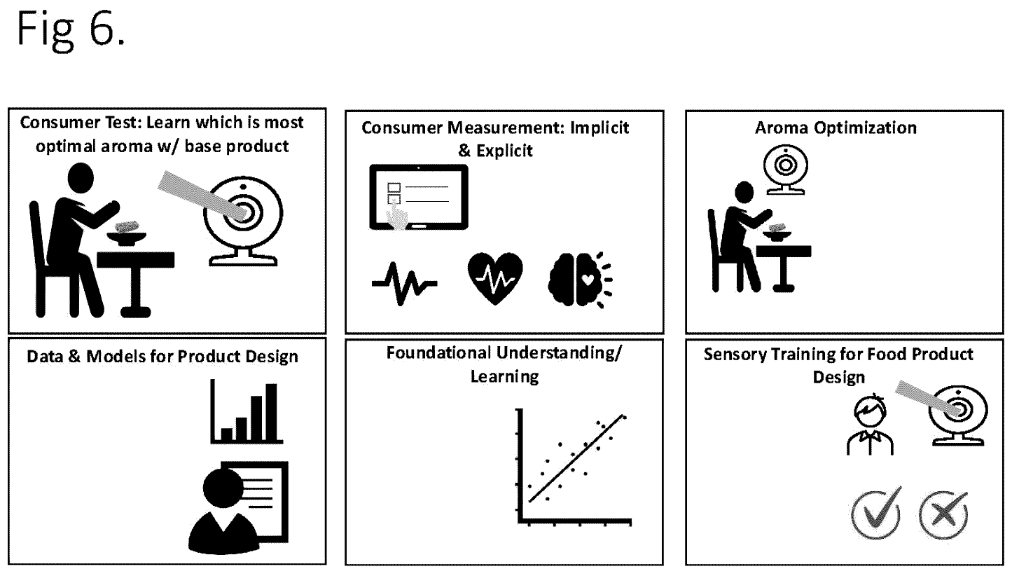

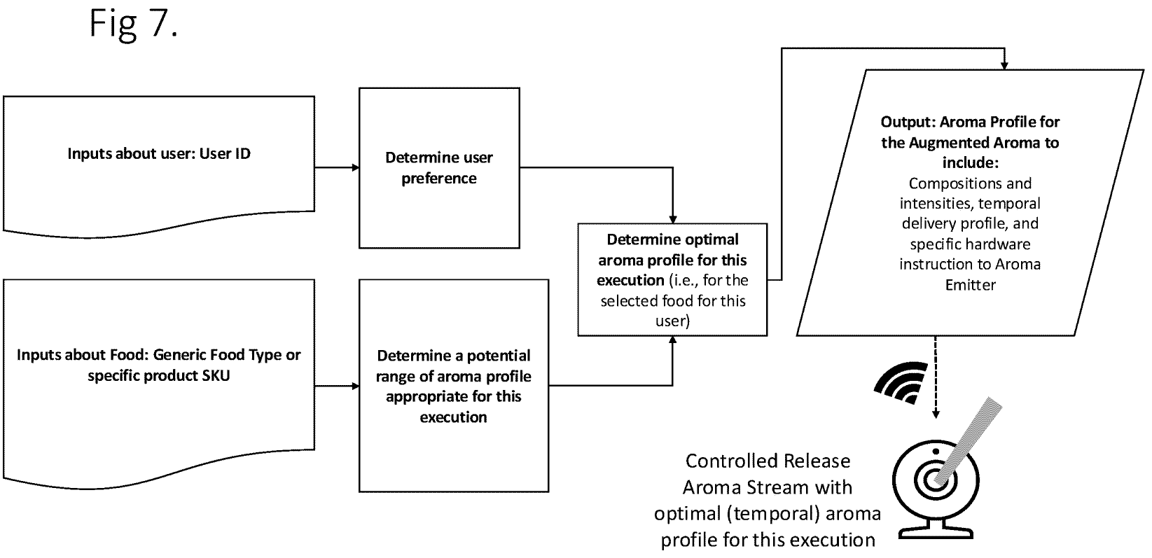

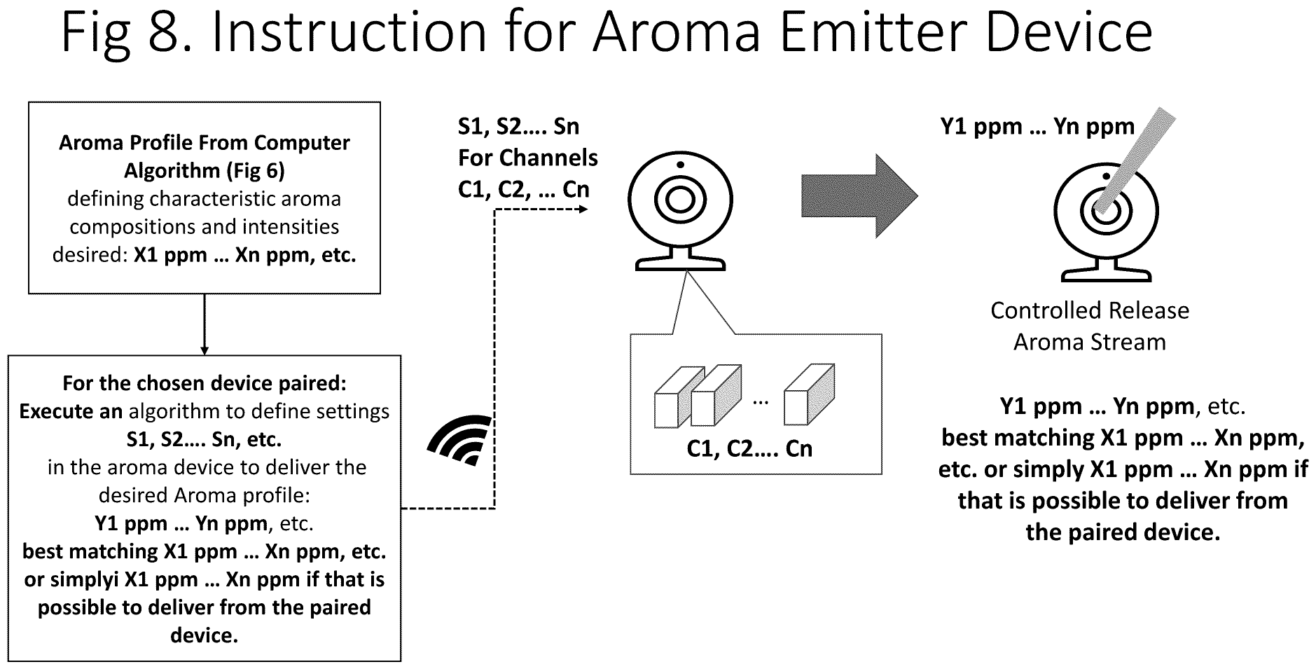

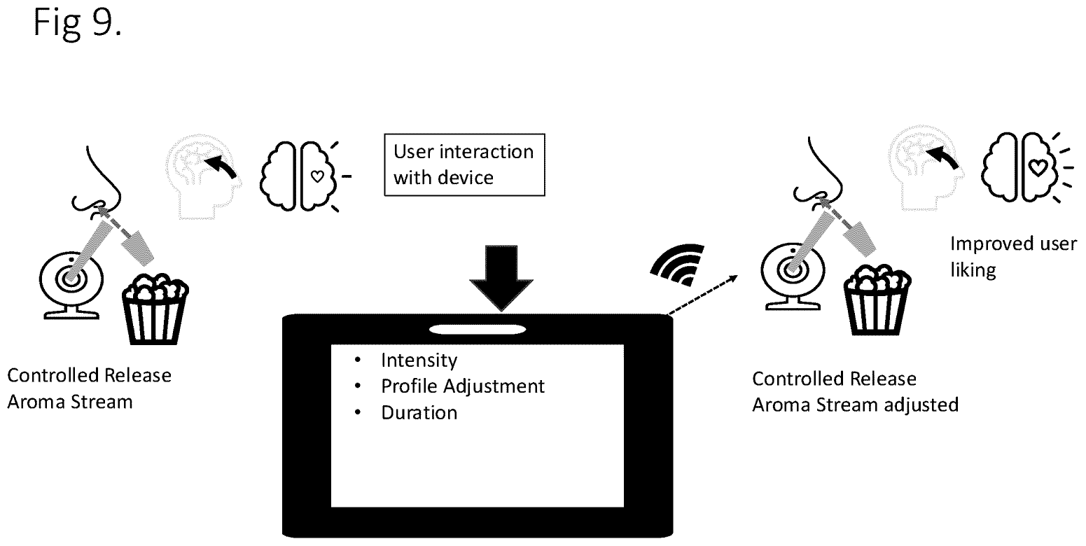

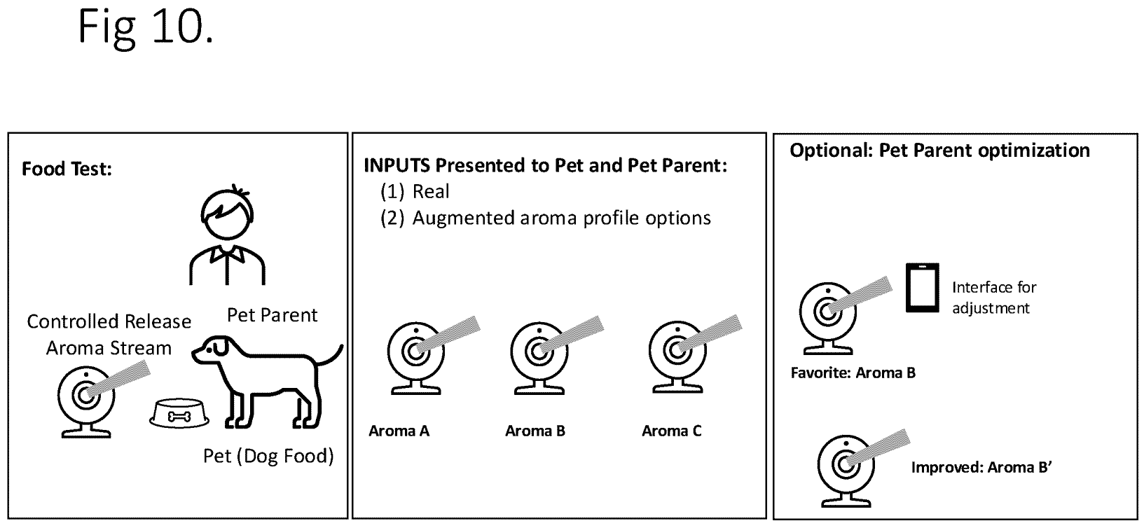

FIG. 11 is a block diagram depicting an illustrative computing device 300, in accordance with embodiments of the disclosure. The computing device 300 may include any type of computing device suitable for implementing aspects of embodiments of the disclosed subject matter. Each of the various components shown and described in the Figures can contain their own dedicated set of computing device components, such as those shown in FIG. 3 and described below.

In embodiments, the computing device 300 includes a bus 310 that, directly and/or indirectly, couples one or more of the following devices: a processor 320, a memory 330, an input/output (I/O) port 340, an I/O component 350, and a power supply 360. Any number of additional components, different components, and/or combinations of components may also be included in the computing device 300.

The bus 310 represents what may be one or more busses (such as, for example, an address bus, data bus, or combination thereof). Similarly, in embodiments, the computing device 300 may include a number of processors 320, a number of memory components 330, a number of I/O ports 340, a number of I/O components 350, and/or a number of power supplies 360. Additionally, any number of these components, or combinations thereof, may be distributed and/or duplicated across a number of computing devices.

In embodiments, the memory 330 includes computer-readable media in the form of volatile and/or nonvolatile memory and may be removable, nonremovable, or a combination thereof. Media examples include random access memory (RAM); read only memory (ROM); electronically erasable programmable read only memory (EEPROM); flash memory; optical or holographic media; magnetic cassettes, magnetic tape, magnetic disk storage or other magnetic storage devices; data transmissions; and/or any other medium that can be used to store information and can be accessed by a computing device. In embodiments, the memory 330 stores computer-executable instructions 370 for causing the processor 320 to implement aspects of embodiments of components discussed herein and/or to perform aspects of embodiments of methods and procedures discussed herein. The memory 330 can comprise a non-transitory computer readable medium storing the computer-executable instructions 370.

The computer-executable instructions 370 may include, for example, computer code, machine-useable instructions, and the like such as, for example, program components capable of being executed by one or more processors 320 (e.g., microprocessors) associated with the computing device 300. Program components may be programmed using any number of different programming environments, including various languages, development kits, frameworks, and/or the like. Some or all of the functionality contemplated herein may also, or alternatively, be implemented in hardware and/or firmware.

According to embodiments, for example, the instructions 370 may be configured to be executed by the processor 320 and, upon execution, to cause the processor 320 to perform certain processes. In certain embodiments, the processor 320, memory 330, and instructions 370 are part of a controller such as an application specific integrated circuit (ASIC), field-programmable gate array (FPGA), and/or the like. Such devices can be used to carry out the functions and steps described herein.

The I/O component 350 may include a presentation component configured to present information to a user such as, for example, a display device, a speaker, and/or the like, and/or an input component such as, for example, a microphone, a joystick, a satellite dish, a wireless device, a keyboard, a pen, a voice input device, a touch input device, a touch-screen device, an interactive display device, a mouse, and/or the like.

The devices and systems described herein can be communicatively coupled via a network, which may include a local area network (LAN), a wide area network (WAN), a cellular data network, via the internet using an internet service provider, and the like.

Aspects of the present disclosure are described with reference to flowchart illustrations and/or block diagrams of methods, devices, systems and computer program products. It will be understood that each block of the flowchart illustrations and/or block diagrams, and combinations of blocks in the flowchart illustrations and/or block diagrams, can be implemented by computer program instructions.

Various modifications and additions can be made to the exemplary embodiments discussed without departing from the scope of the disclosed subject matter. For example, while the embodiments described above refer to particular features, the scope of this disclosure also includes embodiments having different combinations of features and embodiments that do not include all of the described features. Accordingly, the scope of the disclosed subject matter is intended to embrace all such alternatives, modifications, and variations as fall within the scope of the claims, together with all equivalents thereof.

We claim:

1. A method for use with an aroma-emitting device, the method comprising:
obtaining food-product data corresponding to a food product;
selecting, based on the food-product data, an aroma profile for the food product;
emitting, by the aroma-emitting device, an aroma based on the aroma profile;
obtaining modification data corresponding to an instruction to modify the aroma; and
generating a new aroma profile for the food product based on the modification data.

2. The method of claim 1, wherein the modification data indicates an increase or decrease of one or more predetermined flavors.

3. The method of claim 1, wherein the obtaining the food-product data includes reading an optical code corresponding to the food product.

4. A system comprising: an aroma-emitting device; a controller including one or more processors; and one or more computer-readable storage media storing program instructions which, when executed by the one or more processors, are configured to cause the one or more processors to perform a method comprising:
obtaining food-product data corresponding to a food product;
selecting, based on the food-product data, an aroma profile for the food product;
obtaining an indication that an aroma based on the aroma profile is emitted by the aroma-emitting device;
obtaining modification data corresponding to an instruction to modify the aroma; and
generating a new aroma profile for the food product based on the modification data.

5. The system of claim 4, wherein the modification data indicates an increase or decrease of one or more predetermined flavors.

6. The system of claim 4, wherein the obtaining the food-product data includes reading an optical code corresponding to the food product.

7. A method for use with an aroma-emitting device, the method comprising:
emitting, by the aroma-emitting device, a first aroma based on the aroma profile;
emitting, by the aroma-emitting device, a second aroma based on the aroma profile,
where the second aroma is emitted at an intensity lower than the first aroma.

8. The method of claim 7, wherein the first aroma is detectable by a human, and the second aroma is not detectable by a human.

9. The method of claim 8, wherein the second aroma is detectable by pet.
